(19) United States
(12) Patent Application Publication
(10) Pub. No.: US 2005/0197788 A1
(43) Pub. Date: Sep. 8, 2005

(54) Automatic candidate sequencing system and method

(76) Inventor: Peilin Chou, Taipei (TW)

Correspondence Address: TRANSPACIFIC LAW GROUP, 617 NORTH DELAWARE STREET, SAN MATEO, CA, 94401, US

(21) Appl. No.: 10/793,959

(22) Filed: Mar. 4, 2004

(51) Int. Cl. G06F 19/00; G06F 15/18; G01N 33/48; G01N 33/50

(52) U.S. Cl. 702/20; 706/13

(57) ABSTRACT

The automatic candidate sequencing system of this invention comprises: a candidate memory to record at least a group of candidate; each candidate being defined by a plurality of descriptive attribute and parameter thereof; a user interface to allow user to edit descriptive attributes and their parameters, for particular candidate in said group of candidate and to input a result of experiment; a relativity calculation module to calculate value of relativity between each candidate and particular incident according to a filtering function comprising selected descriptive attributes and their weights; a relativity revision module to revise content of said filtering function according to said input of experiment result; and a sequencing module to determine sequence of said candidates according to relativity values calculated by said relativity calculation module.

### FIELD OF THE INVENTION

The present invention relates to method and system for automatic sequencing of candidates, especially to method and system to automatically determine priority of candidates in a list according to factual verification of relativity between several candidates and a particular incident.

### BACKGROUND OF THE INVENTION

In many applications, it is often necessary to establish a list of candidates and to determine the sequence or priority of the candidates according to particular values representing relativity between respective candidates and particular incident. General speaking, the relativity between a candidate and a particular incident is determined or forecast by a person according to selected descriptive attributes and weights of the attributes representing the candidate. However, the reality (the real relation of the selected parameters and the result) and the correctness (compliance of the forecast or the determination to the real result) of the forecast or the determination need to be verified and adjust by experiments. The experiment helps to find out attributers that are more relative, positively or negatively, to the particular incident and to calculate their relativity values, and even inter-relativity among the attributes. The experiment also helps to revise or adjust the function in the calculation of relativity between a candidate and a particular incident. To find out the more relative parameters and a more realistic function, however, requires complicated mental activities of the human being.

Although the mental activities of the human being are of complicated models, it is possible to establish simpler models, utilizing the high-speed operation capability of the computer system, to imitate and to calculate the values of relativity of each candidate in a candidate list and the particular incident.

For example, in the application of direct sales through telephone, when the salesperson is given a list of potential customer, provided with descriptive information of each potential customer, the salesperson would predict according to such descriptive information the chance of success for each potential customer. The salesperson would further determine the order of priority of these potential customers according to the above prediction and start to call the potential customers from those with higher chance of success.

In general, descriptive information that could be provided in a list of potential customer would include: name, age, sex, area of habitation, education, annual income, profession, position, past purchasing history including frequency, quantity and amount, etc. The salesperson can thus determine the possibility of each potential customer to purchase a particular product or service according to past experience or any sales theory. When making such determination, the relativity between a potential customer, as defined by the given descriptive information, and the incident of “purchasing a particular product or service” is estimated based on the descriptive information. The sales person then will sequence all potential customers according to the respective estimated relativity values.

As a matter of fact, it is possible to use a simple computer software to automatically calculate possibility of purchase for candidates belonging to a candidate list and to automatically sequence the candidates. When the possibility of purchase is calculated, a number of candidates with no or low possibility of success may be filtered. Time spent in the sale activity may thus be effectively saved.

However, in using such a sequencing and filtering technology, the sequenced candidate list is generated according to a particular function. As to whether the resulted sequence is correct or not, verification is needed. If result of the verification shows that the sequence is wrong, the function used in sequencing the candidate list shall be revised. Otherwise, the sales costs may go beyond the case when no sequencing is conducted and the sales income may go beneath that.

In conducting the revision, at least three problems will be confronted. First, since the sequence is calculated according to a function or theory that the salesperson believed to be true, it is difficult for the salesperson to realize that the sequence or the function is wrong or needs revisions, until a large quantity of failure examples is accumulated. In other words, the salesperson may not notice the defects of the sequence or the function immediately or before substantial time and costs are invested.

Secondly, when the salesperson finds out that the sequence is not complying with the fact, the sequencing algorithm or function needs to be revised. In revising the sequencing function, some irrelevant attributes of the descriptive information shall be deleted, while some relative attributes of the descriptive information shall be added into the sequencing algorithm or function. Weight values of the attributes in the determination of the sequence may need adjustments. To decide which attribute to be irrelative and which to be relative and how much weight should be given to each attribute in estimating the possibility of success, however, is a difficult judgment. The most difficult part of this revision job rests in that, if the revised theory is later proved to be wrong again, all the revisions and the sales efforts are in vain.

In addition to that, although the calculation of the possibility (relativity) values and the sequencing cost only a short time, the sales activity, however, needs to halt during the calculation and the sequencing procedure. Correctness of sequencing sacrifices the sales efficiency.

### OBJECTIVES OF THE INVENTION

The objective of this invention is to provide a novel automatic candidate sequencing method and system.

Another objective of this invention is to provide a method and system for automatic candidate sequencing, whereby adjustments to the sequencing parameters may be made without the need to wait until a substantial quantity of wrong results have been revealed.

Another objective of this invention is to provide a method and system for automatic sequencing of candidates that automatically adjusts priority sequence of candidates during the process of verification.

Another objective of this invention is to provide a method and system for automatic sequencing of candidates that automatically adjusts priority sequence of candidates during use of the sequence.

### SUMMARY OF THE INVENTION

According to this invention, a method and system for automatic sequencing of candidates is provided. The automatic candidate sequencing system of this invention comprises: a candidate memory to record at least a group of candidate; each candidate being defined by a plurality of descriptive attribute and parameter thereof; a user interface to allow user to edit descriptive attributes and their parameters, for particular candidate in said group of candidate and to input a result of experiment; a relativity calculation module to calculate value of relativity between each candidate and particular incident according to selected descriptive attributes and their weights; a relativity revision module to revise content of said selected descriptive attributes and weights according to said input of experiment result; and a sequencing module to determine sequence of said candidates according to relativity values calculated by said relativity calculation module.

These and other objectives and advantages of this invention may be clearly understood from the detailed description by referring to the following drawings.

### BRIEF DESCRIPTION OF THE DRAWINGS

FIG. 1 shows the systematic diagram of the automatic candidate sequencing system of this invention.

FIG. 2 shows a candidate list of potential customers to be used in a direct sales activity.

FIG. 3 shows a candidate list that may be used in the automatic candidate sequencing system of this invention.

FIG. 4 illustrates the flowchart in the calculation of possibility of purchase as used by the automatic candidate sequencing system of this invention.

FIG. 5 illustrates the flowchart in the revision of filtering function, as used by the relativity revision module of this invention.

FIG. 6 illustrates the flowchart of the automatic candidate sequencing method of this invention.

### DETAILED DESCRIPTION OF THE INVENTION

Detailed description of the method and system for automatic sequencing of candidates will be given in the followings by referring to the drawings.

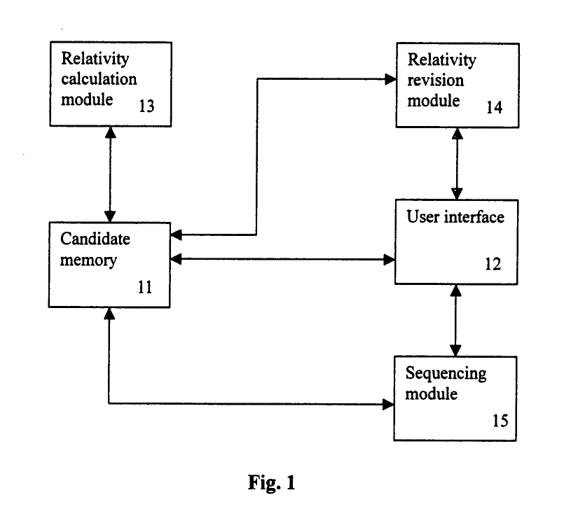

FIG. 1 shows the systematic diagram of the automatic candidate sequencing system of this invention. As shown in this figure, the automatic candidate sequencing system of this invention comprises: a candidate memory 11 to record at least a group of candidate and corresponding plurality of descriptive attribute and parameter defining each candidate; a user interface 12 to allow user to edit descriptive attributes and parameters, for particular candidate in said group of candidate, and to input a result of experiment; a relativity calculation module 13 to calculate value of relativity between each candidate and a particular incident according to selected descriptive attributes and their weights; a relativity revision module 14 to revise content of said selected descriptive attributes and their weights according to said input of experiment result; and a sequencing module 15 to determine sequence of said candidates according to relativity values calculated by said relativity calculation module.

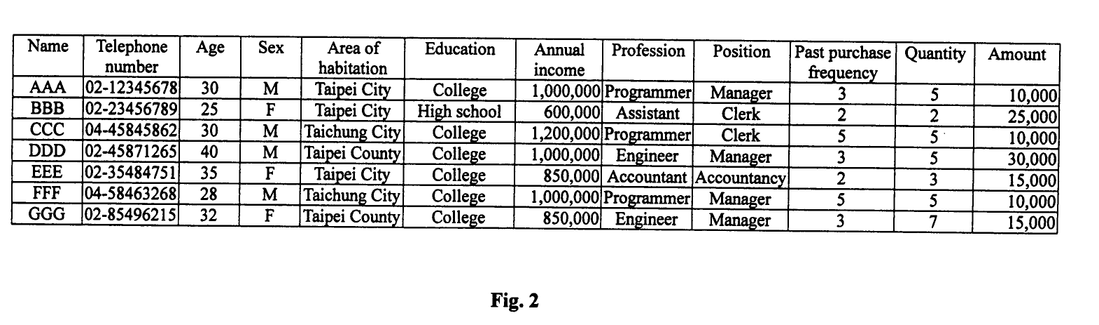

In the followings, the automatic candidate sequencing system of this invention will be described by taking the case of direct sales through telephone as example. FIG. 2 shows a candidate list that is often used in the direct sales activity. The candidate list comprises a plurality of potential customers and each candidate is connected to descriptive information including: name, telephone number, age, sex, area of habitation, education, annual income, profession, position, past purchase history such as frequency, quantity and amount of purchase and other useful or not useful information. In the list, descriptive information such as name, age etc., may be treated as attributes defining the potential customers. It is thus possible to use a function to include all the descriptive attributes as parameters of a function to define the potential customers. For example:

The age of the customers may be divided into groups. Each group is given a parameter value. It is also possible to give every age an attribute and any customer belonging to that age is given a parameter of 1, otherwise, 0. Attributes such as area of habitation, income etc. may also be given attributes and parameters in a similar way. In addition, it is also possible to give parameters according to distance of habitation, level of income etc., of the customer.

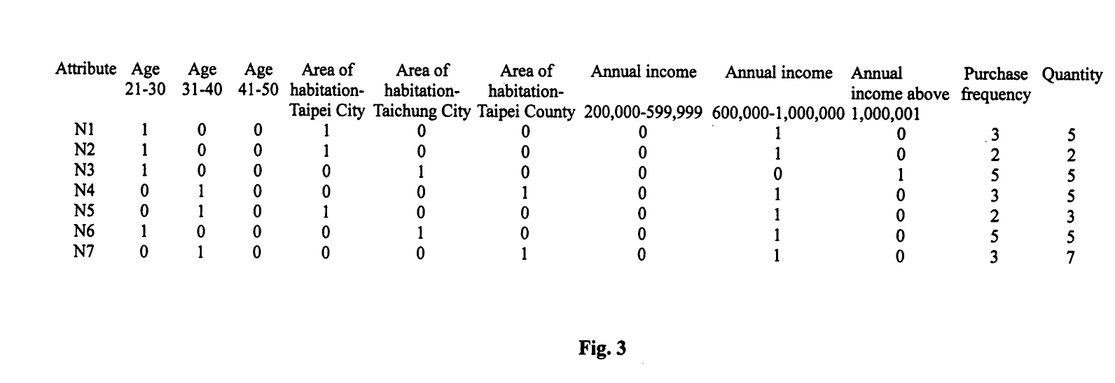

After the above-said or other method of conversion, the descriptive information of all candidates (customers) is defined a plurality of attributes and a parameter is given to each attribute. The resulted candidate list is shown in FIG. 3. FIG. 3 shows a candidate list that may be used in the automatic candidate sequencing system of this invention. FIG. 3 shows that a parameter is given to each attribute. It is also possible to generate parameter values using functions or other algorithms using the descriptive information as shown in FIG. 2. As a result, every potential customer Ni may be represented by the following function:
Nᵢ=(a₁,p₁),(a₂,p₂), . . . ,(aₙ,pₙ) (1)
wherein Ni represents the i^{th} candidate, a represents attribute and p represents parameter of the attribute.

In the present invention, the candidate list memory 11 is used to store a plurality of candidate lists, each candidate list including a plurality of candidates defined by attributes and parameters as shown in Equation (1). All the attributes and the parameters may be input, adjusted or deleted by user in the user interface 12.

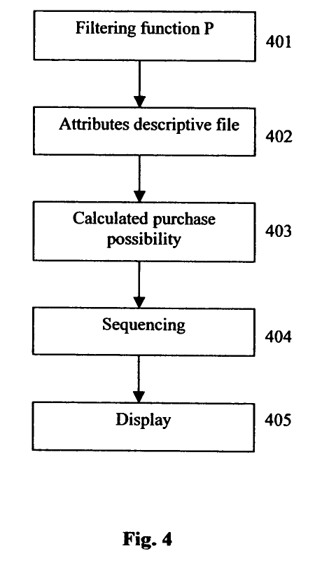

In this embodiment, the relativity calculation module 13 is used to calculate the relativity between each candidate and the result of the candidate purchasing a particular product or service. In other words, it calculates the possibility of particular potential customers to purchase a particular product or service. In this invention, the possibility or relativity is calculated according to selected attributes and weights given to the selected attributes. The method in the calculation of such possibility will be described by referring to FIG. 4. FIG. 4 illustrates the flowchart in the calculation of possibility of purchase as used by the automatic candidate sequencing system of this invention.

As shown in FIG. 4, in order to calculate the possibility of a potential customer to purchase a particular product or service, first at 401 a filtering function P is generated, as follows:
P=(b₁w₁),(b₂,w₂), . . . ,(bₘ,wₘ) (2)
wherein b represents filtering attribute and w represents its weight. P denotes in general description or prediction of a customer that would purchase a particular product or service. As a result, the content of P may be generated manually by user before the filtering function is used to search or sequence potential customers in a candidate list. The content of P may also be generated automatically by a computer according to past sequencing experiences.

Then at 402 the filtering function is used to search potential customers in a candidate list. Method of search includes comparison of attributes and parameter values of each candidate with attribute of the filtering function and listing attributes that exist in both the candidate descriptive function Ni and the filtering function P, with parameters of the descriptive attributes to be other than 0. The list becomes a new descriptive function of the particular potential customers.

Later, at 403, possibility of purchase for particular candidates is calculated, according to the following equation:
Pᵢ=Σwᵢ×pᵢ,∀aᵢ=bᵢ (3)
wherein P represents possibility of success.

After the calculation, the predicted values of possibility of success are obtained. At 404 the sequencing module 15 determines priority or sequence of the candidates according to the obtained values. In doing so, candidates with greater values of relativity are listed in prior positions and at 405 the result is shown in the user interface 12. Thereby the salesperson may conduct direct sales activity by making phone calls to the listed potential customers, following the sequence displayed in the user interface 12.

During the direct sale activity, depending on the purposes of the telephone conversation, standards for “success” and “failure” may be determined. For example, if the purpose of the telephone conversation is to introduce to the potential customers a new product, when the potential customer expresses his/her interests in the product and agrees to receive further information, the conversation may be deemed “successful”; otherwise the conversation is “failed”. When the salesperson completes a telephone conversation, the result, success or failure, is input into the system through the user interface 12. Although a successful telephone conversation does not necessarily mean the sequence of the candidate list is correct, a failed telephone conversation could mean that the sequence of the candidate list is defected. In the present invention, the input of a “failure” message triggers the relativity revision module 14 to enter into the relativity revision mode.

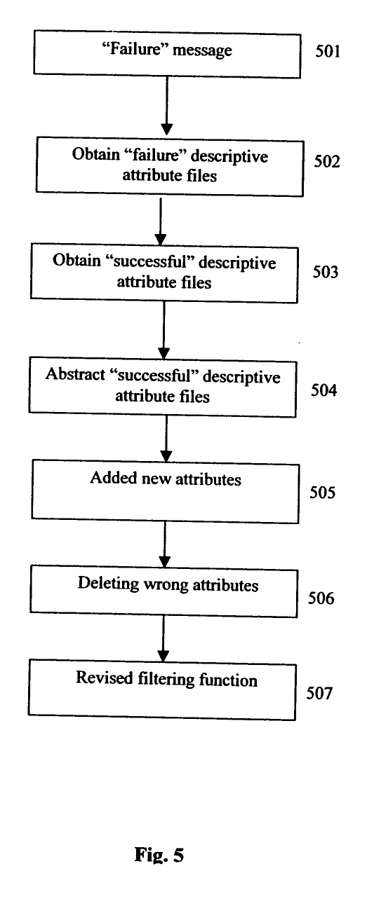

The processing of the relativity revision mode will be described by referring to FIG. 5. FIG. 5 illustrates the flowchart in the revision of the filtering function, as used by the relativity revision module of this invention. As shown in this figure, at 501 the user inputs a “failure” message and the relativity revision mode is triggered. At 502 the relativity revision module 14 obtains the descriptive attribute file corresponding to the candidate that is labeled previously as “failed” by user. At 503 the relativity revision module 14 obtains the descriptive attribute files of all candidates that have been labeled as “successful” and abstracts at 504 descriptive attributes that exist or have a parameter other than 0 in at least a ratio of the “successful” candidates, but are not include in the filtering function. These descriptive attributes are added into the filtering function at 505, with a parameter value being set to each attribute. At 506 the relativity revision module 14 obtains descriptive attributes that exist in the filtering function but do not exist or have a parameter of 0 in at least a ratio of the “successful” candidates and deletes these descriptive parameters from the filtering function. At 507 the revised filtering function is stored in the memory 11 for further use.

After the filtering function is revised, the relativity calculation module 13 recalculates the possibility of success of the rest candidates. Thereafter, the sequencing module 15 determines the priority sequence of the candidates and displays information of candidates with higher possibility of success in the user interface 12. The relativity revision module 14 will be triggered whenever a “failure” message is input by the user.

As described above, revision of the filtering function and calculation of the possibility of success are not complicated. The filtering function may be revised and results of the re-sequencing may be displayed immediately after a “failure” message is input. If the automatic candidate sequencing system is used in a direct sales station wherein a group of salespersons is conducting telephone conversation at the same time, the sequence of the potential customers as displayed in the displaying screen of all salespersons may be changed immediately after the filtering function is revised following a “failure” message input by any one of the salespersons. As a result, the filtering function is dynamically revised and all salespersons can share the most updated sequence of candidate in a real-time manner. Direct sales can thus be made effectively.

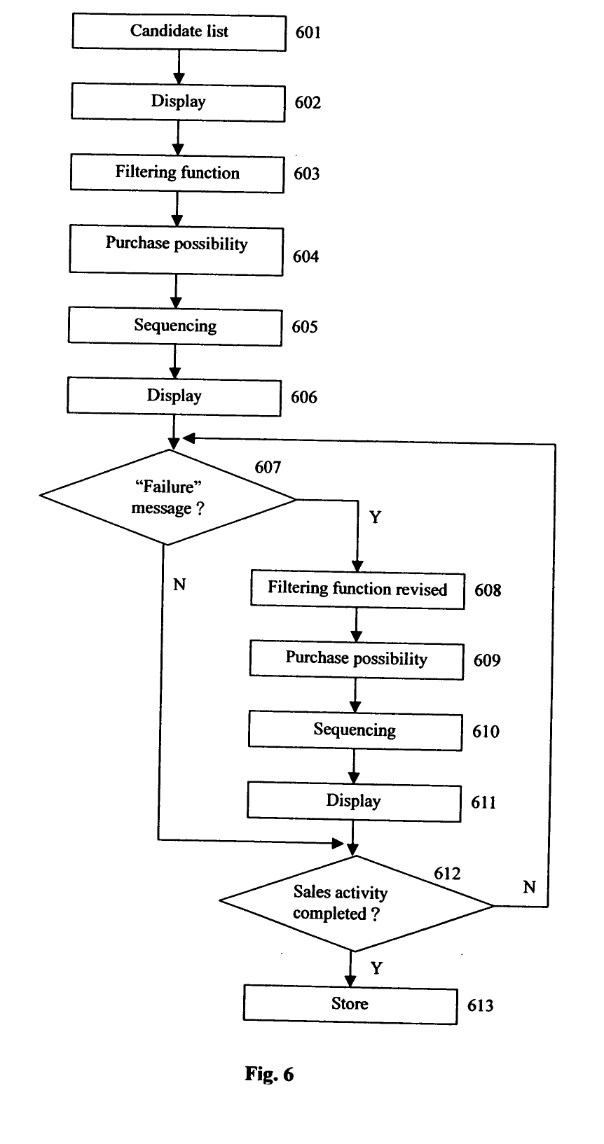

The automatic candidate sequencing method of this invention will be described by referring to FIG. 6. FIG. 6 illustrates the flowchart of the automatic candidate sequencing method of this invention. As shown in this figure, at 601 a candidate list comprising a plurality of potential customer is obtain and displayed in the user interface 12 at 602. At 603 the system generates a filter function to be used to predict the possibility of a potential customer to purchase a particular product. At 604 the relativity calculation module 13 uses Formula (3) to calculate the possibility of each potential customer to purchase the product. At 605 the sequencing module 15 determines the sequence of the potential customers according to the possibility values obtained by the relativity calculation module 13 and displays information of a predetermined number of potential customers, who have the highest possibility values at the user interface 12 at 606. Then at 607 a “failure” message is input by user. At 608 the relativity revision module 14 revises the filtering function. At 609 the relativity calculation module 13 calculates the possibility for the remaining potential customers to purchase the product, using to the revised filtering function. At 610 the sequencing module sequences the remaining potential customers according to the new possibility values. At 611 the potential customers with higher possibility values are listed in the user interface 12. At 612 the system determines whether the direct sales activity is completed. If not, the process goes back to step 607 to determines whether any “failure” message is obtained. If yes, the process goes to step 608 and steps 608-612 are repeated. Otherwise, the process returns to step 612. If the determination at step 612 is yes, results of the direct sales activity are stored into the candidate list file at 613.

The method and system for automatic candidate sequencing of this invention dynamically revises the sequencing process of the candidates during their operation. If a sequence of candidate does not comply with the factual situation, such defect may be easily detected without the need to wait until a substantial number of wrong results is generated. During the adjustment procedure, the operation needs not be stopped to wait for the adjustment. No other technology in the conventional art provides such advantageous functions.

In the above description, the application of this invention in the filed of direct sales is taken for illustration purpose. Anyone skilled in the art may understand that the present invention may be applied to sequencing of candidate of any type, feature and characteristics.

As the present invention has been shown and described with reference to preferred embodiments thereof, those skilled in the art will recognize that the above and other changes may be made therein without departing form the spirit and scope of the invention.

1. An automatic candidate sequencing system, comprising:
a candidate memory to record at least a group of candidates, each candidate being defined by a series of descriptive attributes and their parameters;
a user interface to allow user to edit descriptive attributes and their parameters defining particular candidate in said group and to input a result of experiment, which result comprises at least “failure” and “success”;
a relativity calculation module to calculate relativity between each candidate and a particular incident according to a filtering function comprising selected descriptive attributes and weights given to said selected descriptive attributes;
a relativity revision module to revise content of said filtering function upon the input message of a “failure” result; and
a sequencing module to determine sequence of said candidates according to relativity values calculated by said relativity calculation module.

2. The automatic candidate sequencing system according to claim 1, wherein each candidate is defined by the series of descriptive attributes and their respective parameters, as follows:
Nᵢ=(a₁,p₁),(a₂,p₂), . . . ,(aₙ,pₙ) (1)
wherein Nᵢ represents the i^{th} candidate, a represents descriptive attribute and p represents parameter of said attribute; and
wherein said relativity calculation module uses the filtering function P:
P=(b₁,w₁),(b₂,w₂), . . . ,(bₘ,wₘ) (2)
wherein b represents filtering attribute and w represents its weight;
to calculate relativity between said Ni candidate and said particular incident according to the following equation:
Pᵢ=Σwᵢ×p₁,∀aᵢ=bᵢ (3)
wherein Pi represents relativity.

3. The automatic candidate sequencing system according to claim 2, wherein said relativity revision module revises said filtering function according to the following steps:
obtaining a “failure” message;
obtaining descriptive attributes and their parameters of all candidates in connection with a “success” result;
obtaining descriptive attributes that have parameter other than 0 in at least a first ratio of said “success” candidates but are not included in said filtering function;
adding any so obtained descriptive attribute into said filtering function and giving said obtained descriptive attribute a weight value;
obtaining descriptive attributes that has parameter of 0 in at least a second ratio of said “success” candidates; and
deleting any so obtained descriptive attribute that is included in said filtering function from said filtering function.

4. An automatic candidate sequencing method, comprising the steps of:
obtaining a list of candidates, each candidate being defined by a series of descriptive attributes and their respective parameters, as follows:
Nᵢ=(a₁,p₁),(a₂,p₂), . . . ,(aₙ,pₙ) (1)
wherein Nᵢ represents the i^{th} candidate, a represents descriptive attribute and p represents parameter of said attribute; and
generating a filtering function P represented by:
P=(b₁,w₁),(b₂,w₂), . . . ,(bₘ,wₘ) (2)
wherein b represents filtering attribute and w represents its weight;
calculating relativity between said Ni candidate and a particular incident using said filtering function P; and
sequencing said candidates according to result of said calculation.

5. The automatic candidate sequencing method according to claim 4, wherein said relativity is calculated according to the following equation:
Pᵢ=Σwᵢ×pᵢ,∀aᵢ=bᵢ (3)
wherein Pi represents relativity.

6. The automatic candidate sequencing method according to claim 4, further comprising a process of revising said filtering function, comprising the steps of:
obtaining descriptive attributes and their parameters of all candidates in connection with a “success” result;
obtaining descriptive attributes that have parameter other than 0 in at least a first ratio of said “success” candidates;
adding any so obtained descriptive attribute that is not contained in said filtering function into said filtering function and giving said obtained descriptive attribute a weight value;
obtaining descriptive attributes that has parameter of 0 in at least a second ratio of said “success” candidates; and
deleting any so obtained descriptive attribute that is included in said filtering function from said filtering function.
